(19) United States
(12) Patent Application Publication
(10) Pub. No.: US 2014/0194768 A1
(43) Pub. Date: Jul. 10, 2014

(54) Method And System To Calculate qEEG

(71) Applicant: PERSYST DEVELOPMENT CORPORATION, (US)

(72) Inventors: Nicolas Nierenberg, La Jolla, CA (US); Scott B. Wilson, Del Mar, CA (US); Mark L. Scheuer, Wexford, PA (US)

(73) Assignee: PERSYST DEVELOPMENT CORPORATION, San Diego, CA (US)

(21) Appl. No.: 13/830,742

(22) Filed: Mar. 14, 2013

Related U.S. Application Data
(63) Continuation-in-part of application No. 13/620,855, filed on Sep. 15, 2012.
(60) Provisional application No. 61/536,236, filed on Sep. 19, 2011.

(51) Int. Cl. A61B 5/04; A61B 5/0476; A61B 5/00

(52) U.S. Cl. CPC A61B 5/04012; A61B 5/7257; A61B 5/7207; A61B 5/0476
USPC 600/544

(57) ABSTRACT

A system and method for calculating a quantitative EEG is disclosed herein. The present invention achieves a level of artifact reduction that the QEEG is now practical on a continuous monitoring basis since artifact reduction is continuously applied to an EEG recording.

### CROSS REFERENCE TO RELATED APPLICATION

The Present application is a continuation-in-part application of U.S. patent application Ser. No. 13/620,855, filed on Sep. 15, 2012, which claims priority to U.S. Provisional Patent Application No. 61/536,236, filed on Sep. 19, 2011, now abandoned, both of which are hereby incorporated by reference in their entireties.

### STATEMENT REGARDING FEDERALLY SPONSORED RESEARCH OR DEVELOPMENT

Not Applicable

### BACKGROUND OF THE INVENTION

1. Field of the Invention

The present invention generally relates to a method and system for calculating a quantitative EEG.

2. Description of the Related Art

An electroencephalogram (“EEG”) is a diagnostic tool that measures and records the electrical activity of a person's brain in order to evaluate cerebral functions. Multiple electrodes are attached to a person's head and connected to a machine by wires. The machine amplifies the signals and records the electrical activity of a person's brain. The electrical activity is produced by the summation of neural activity across a plurality of neurons. These neurons generate small electric voltage fields. The aggregate of these electric voltage fields create an electrical reading which electrodes on the person's head are able to detect and record. An EEG is a superposition of multiple simpler signals. In a normal adult, the amplitude of an EEG signal typically ranges from 1 micro-Volt to 100 micro-Volts, and the EEG signal is approximately 10 to 20 milli-Volts when measured with subdural electrodes. The monitoring of the amplitude and temporal dynamics of the electrical signals provides information about the underlying neural activity and medical conditions of the person.

An EEG is performed to: diagnose epilepsy; verify problems with loss of consciousness or dementia; verify brain activity for a person in a coma; study sleep disorders, monitor brain activity during surgery, and additional physical problems.

Multiple electrodes (typically 17-21, however there are standard positions for at least 70) are attached to a person's head during an EEG. The electrodes are referenced by the position of the electrode in relation to a lobe or area of a person's brain. The references are as follows: F=frontal; Fp=frontopolar; T=temporal; C=central; P=parietal; 0=occipital; and A=auricular (ear electrode). Numerals are used to further narrow the position and “z” points relate to electrode sites in the midline of a person's head. An electrocardiogram (“EKG”) may also appear on an EEG display.

The EEG records brain waves from different amplifiers using various combinations of electrodes called montages. Montages are generally created to provide a clear picture of the spatial distribution of the EEG across the cortex. A montage is an electrical map obtained from a spatial array of recording electrodes and preferably refers to a particular combination of electrodes examined at a particular point in time.

In bipolar montages, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode.

Reference montages are good for determining the true amplitude and morphology of a waveform. For temporal electrodes, CZ is usually a good scalp reference.

Being able to locate the origin of electrical activity (“localization”) is critical to being able to analyze the EEG. Localization of normal or abnormal brain waves in bipolar montages is usually accomplished by identifying “phase reversal,” a deflection of the two channels within a chain pointing to opposite directions. In a referential montage, all channels may show deflections in the same direction. If the electrical activity at the active electrodes is positive when compared to the activity at the reference electrode, the deflection will be downward. Electrodes where the electrical activity is the same as at the reference electrode will not show any deflection. In general, the electrode with the largest upward deflection represents the maximum negative activity in a referential montage.

Some patterns indicate a tendency toward seizures in a person. A physician may refer to these waves as “epileptiform abnormalities” or “epilepsy waves.” These include spikes, sharp waves, and spike-and-wave discharges. Spikes and sharp waves in a specific area of the brain, such as the left temporal lobe, indicate that partial seizures might possibly come from that area. Primary generalized epilepsy, on the other hand, is suggested by spike-and-wave discharges that are widely spread over both hemispheres of the brain, especially if they begin in both hemispheres at the same time.

There are several types of brain waves: alpha waves, beta waves, delta wave, theta waves and gamma waves. Alpha waves have a frequency of 8 to 12 Hertz (“Hz”). Alpha waves are normally found when a person is relaxed or in a waking state when a person's eyes are closed but the person is mentally alert. Alpha waves cease when a person's eyes are open or the person is concentrating. Beta waves have a frequency of 13 Hz to 30 Hz. Beta waves are normally found when a person is alert, thinking, agitated, or has taken high doses of certain medicines. Delta waves have a frequency of less than 3 Hz. Delta waves are normally found only when a person is asleep (non-REM or dreamless sleep) or the person is a young child. Theta waves have a frequency of 4 Hz to 7 Hz. Theta waves are normally found only when the person is asleep (dream or REM sleep) or the person is a young child. Gamma waves have a frequency of 30 Hz to 100 Hz. Gamma waves are normally found during higher mental activity and motor functions.

The following definitions are used herein.

“Amplitude” refers to the vertical distance measured from the trough to the maximal peak (negative or positive). It expresses information about the size of the neuron population and its activation synchrony during the component generation.

The term “analogue to digital conversion” refers to when an analogue signal is converted into a digital signal which can then be stored in a computer for further processing. Analogue signals are “real world” signals (e.g., physiological signals such as electroencephalogram, electrocardiogram or electrooculogram). In order for them to be stored and manipulated by a computer, these signals must be converted into a discrete digital form the computer can understand.

“Artifacts” are electrical signals detected along the scalp by an EEG, but that originate from non-cerebral origin. There are patient related artifacts (e.g., movement, sweating, ECG, eye movements) and technical artifacts (50/60 Hz artifact, cable movements, electrode paste-related).

The term “differential amplifier” refers to the key to electrophysiological equipment. It magnifies the difference between two inputs (one amplifier per pair of electrodes).

“Duration” is the time interval from the beginning of the voltage change to its return to the baseline. It is also a measurement of the synchronous activation of neurons involved in the component generation.

“Electrode” refers to a conductor used to establish electrical contact with a nonmetallic part of a circuit. EEG electrodes are small metal discs usually made of stainless steel, tin, gold or silver covered with a silver chloride coating. They are placed on the scalp in special positions.

“Electrode gel” acts as a malleable extension of the electrode, so that the movement of the electrodes leads is less likely to produce artifacts. The gel maximizes skin contact and allows for a low-resistance recording through the skin.

The term “electrode positioning” (10/20 system) refers to the standardized placement of scalp electrodes for a classical EEG recording. The essence of this system is the distance in percentages of the 10/20 range between Nasion-Inion and fixed points. These points are marked as the Frontal pole (Fp), Central (C), Parietal (P), occipital (O), and Temporal (T). The midline electrodes are marked with a subscript z, which stands for zero. The odd numbers are used as subscript for points over the left hemisphere, and even numbers over the right

“Electroencephalogram” or “EEG” refers to the tracing of brain waves, by recording the electrical activity of the brain from the scalp, made by an electroencephalograph.

“Electroencephalograph” refers to an apparatus for detecting and recording brain waves (also called encephalograph).

“Epileptiform” refers to resembling that of epilepsy.

“Filtering” refers to a process that removes unwanted frequencies from a signal.

“Filters” are devices that alter the frequency composition of the signal.

“Montage” means the placement of the electrodes. The EEG can be monitored with either a bipolar montage or a referential one. Bipolar means that there are two electrodes per one channel, so there is a reference electrode for each channel. The referential montage means that there is a common reference electrode for all the channels.

“Morphology” refers to the shape of the waveform. The shape of a wave or an EEG pattern is determined by the frequencies that combine to make up the waveform and by their phase and voltage relationships. Wave patterns can be described as being: “Monomorphic”. Distinct EEG activity appearing to be composed of one dominant activity. “Polymorphic”. distinct EEG activity composed of multiple frequencies that combine to form a complex waveform. “Sinusoidal”. Waves resembling sine waves. Monomorphic activity usually is sinusoidal. “Transient”. An isolated wave or pattern that is distinctly different from background activity.

“Spike” refers to a transient with a pointed peak and a duration from 20 to under 70 msec.

The term “sharp wave” refers to a transient with a pointed peak and duration of 70-200 msec.

The term “neural network algorithms” refers to algorithms that identify sharp transients that have a high probability of being epileptiform abnormalities.

“Noise” refers to any unwanted signal that modifies the desired signal. It can have multiple sources.

“Periodicity” refers to the distribution of patterns or elements in time (e.g., the appearance of a particular EEG activity at more or less regular intervals). The activity may be generalized, focal or lateralized.

An EEG epoch is an amplitude of a EEG signal as a function of time and frequency.

Quantitative EEG (QEEG) was been used for some time in the analysis of EEG. The most common use is for time compressed graphical output using FFT. This type of graphical output can be interpreted by a human reader to show, for example an overview of a long period EEG in the frequency range. While a single page of EEG might display ten seconds of data, a page of QEEG might display minutes or even hours.

QEEG can also be used to produce time averaged results with a single numeric value at a given point in time. This could be as simple as an average amplitude. Or it could be a computation limited to waves in a single frequency range.

QEEG can be limited to a subset of the number of recorded channels. In this way the computation is reflective of activity in a hemisphere, or smaller portion of the brain.

Also the computation might be computed as a relative value of two subsets of the channels or two different frequency ranges. The idea being that a change in these relative values could be diagnostically significant.

There has been a great deal of academic interest in using QEEG to interpret the EEG. The concept is that it might be much less subjective and quicker than reviewing the underlying waveforms. Also patterns may emerge over time that are difficult if not impossible to see otherwise.

One example is the diagnosis of stroke. It is believed that when a stroke begins that changes in brain activity are almost immediately reflected in an EEG. This will occur in many cases significantly before there are clinical symptoms. Therefore, there is great interest in continuous monitoring of patients at risk of stroke to provide early diagnosis and treatment.

However the obstacles to continuous monitoring are significant. First it is very labor intensive to continually monitor the raw EEG signals. Second the types of small relative changes reflective of stroke are very difficult to observe, particularly when presented with only ten seconds of data at a time. QEEG could be a solution to this and there has been significant on-going research trying to determine what sort of computation might show the types of changes reflective of a stroke. Work in this area has been largely thwarted, however by the very large presence of artifact in EEG.

In scalp EEG signals from artifact such as muscle, eye movement, and poor electrical contact by an electrode can overwhelm the signals for the brain. An expert reviewer learns to ignore these artifacts and focus on the artifact free portions, however QEEG doesn't have this luxury and all the signals are included in the computation. The result is that QEEG often reflects artifact as much or more than it reflects brain activity. This is, of course, problematic when producing graphical results, but in that case an expert reviewer again might be able to discern patterns stemming from brain activity. However in the case of discrete values being computed for the purpose of diagnosis it is a very large issue. For this reason researchers frequently try to pick relatively artifact free segments to do computations, but this is, of course, not available in clinical practice.

Thus, there is a need for QEEG that contains the full signal but greatly reduced artifacts, especially in a clinical setting.

### BRIEF SUMMARY OF THE INVENTION

The solution is to computationally remove many of the artifacts present in a record prior to QEEG processing. In this way the signal to noise ratio can be dramatically improved, and the resulting QEEG computation will reflect cerebral activity. At this point it is then possible to both determine what types of QEEG will be effective in diagnosis, and to use it clinically.

There has been research and discussion in the field that it may be possible to anticipate clinical symptoms of stroke using calculated measures of EEG (QEEG).

One of the primary issues with doing anticipating clinical symptoms of a stroke using QEEG was that the artifact when mixed into the cerebral signal produced unreliable quantitative values. The present invention achieves a level of artifact reduction that the QEEG is now practical on a continuous monitoring basis.

As an example in stroke diagnosis a physician could begin continuous monitoring of one or more QEEg parameters that have been determined to be diagnostic. Having established a baseline the physician could set ranges for these parameters and if the QEEG moved outside these ranges the staff would be alerted to a possible stroke. In a more automated implementation a system might determine the baseline and set ranges automatically, or it might use an intelligent system such as neural networks to determine the QEEG to use, and a set of changes that represent a stroke.

A stroke is only a single example, and many other conditions affecting cerebral activity can diagnosed in this manner.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is an image of a quantitative EEG.

FIG. 2 is a block diagram of a system for calculating a quantitative EEG.

FIG. 3 is a map for electrode placement for an EEG.

FIG. 4 is a detailed map for electrode placement for an EEG.

FIG. 5 is an illustration of a CZ reference montage.

FIG. 6 is an illustration of an EEG recording containing a seizure, a muscle artifact and an eye movement artifact.

FIG. 7 is an illustration of the EEG recording of FIG. 6 with the muscle artifact removed.

FIG. 8 is an illustration of the EEG recording of FIG. 7 with the eye movement artifact removed.

FIG. 9 is a flow chart for a method for calculating a quantitative EEG.

FIG. 10 is a flow chart method for calculating a quantitative EEG.

FIG. 11 is a block diagram of a system for calculating a quantitative EEG.

### DETAILED DESCRIPTION OF THE INVENTION

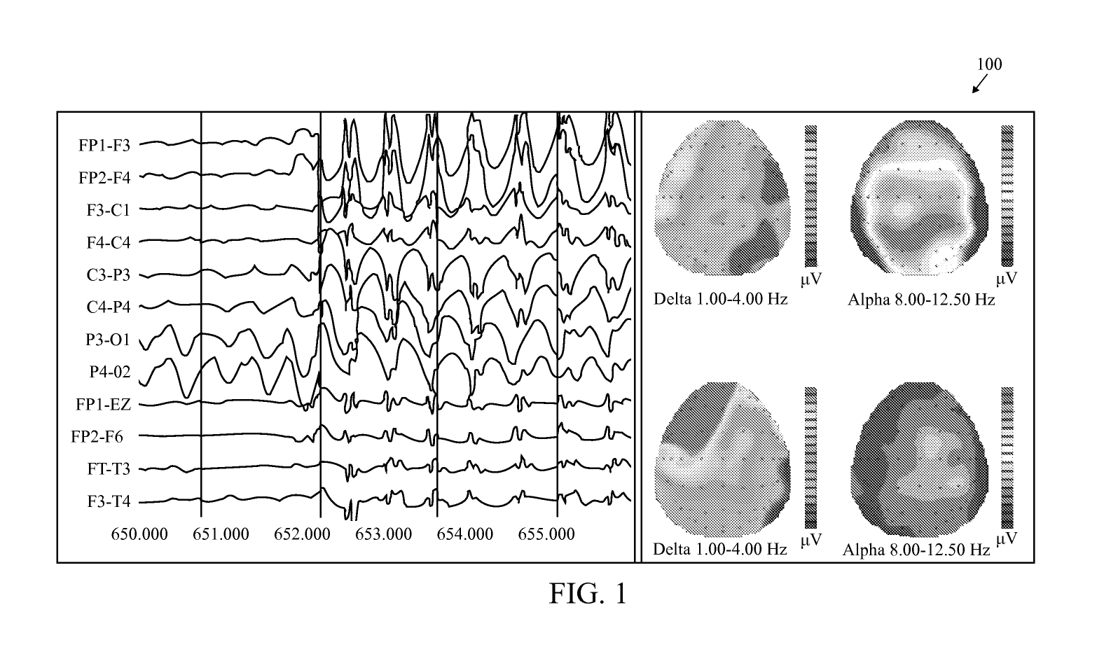

An image 100 of a quantitative EEG (“qEEG”) is shown in FIG. 1. The method and system allows for a qEEG to be generated from an artifact reduced EEG recording without having to remove portions of the EEG recording to prevent artifacts from influencing the qEEG.

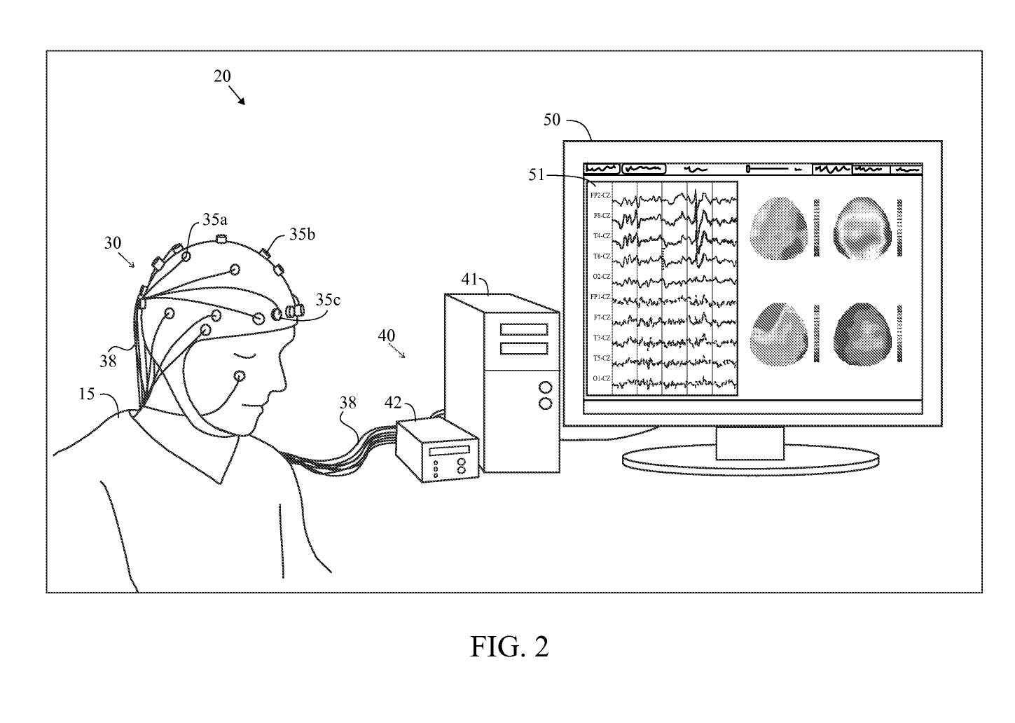

FIG. 2 illustrates a system 20 for calculating a quantitative EEG. A patient 15 wears an electrode cap 31, consisting of a plurality of electrodes 35a-35c, attached to the patient's head with wires 38 from the electrodes 35 connected to an EEG machine component 40 which consists of an amplifier 42 for amplifying the signal to a computer 41 with a processor, which is used to analyze the signals from the electrodes 35 and generate an EEG recording 51 and a qEEG, which can be viewed on a display 50. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is used to generate a qEEG.

An additional description of analyzing EEG recordings is set forth in Wilson et al., U.S. patent application Ser. No. 13/620,855, filed on Sep. 15, 2012, for a Method And System For Analyzing An EEG Recording, which is hereby incorporated by reference in its entirety.

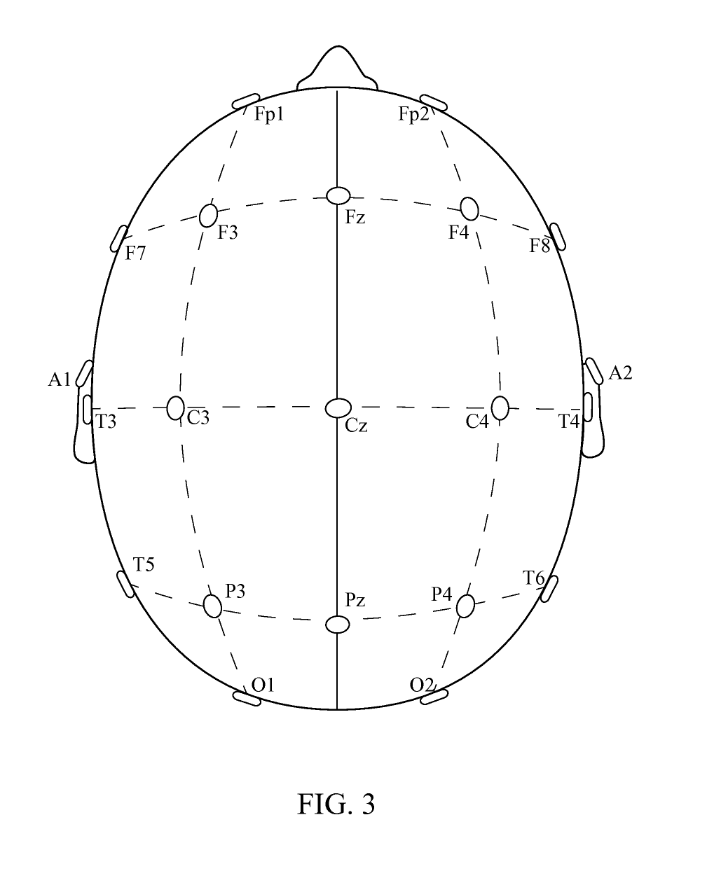

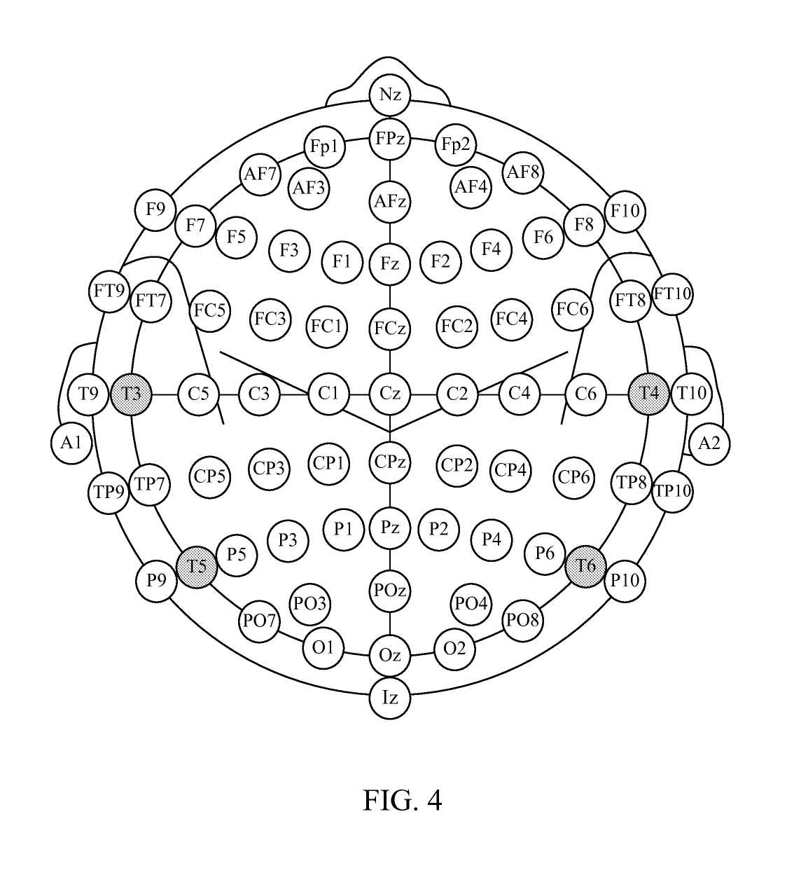

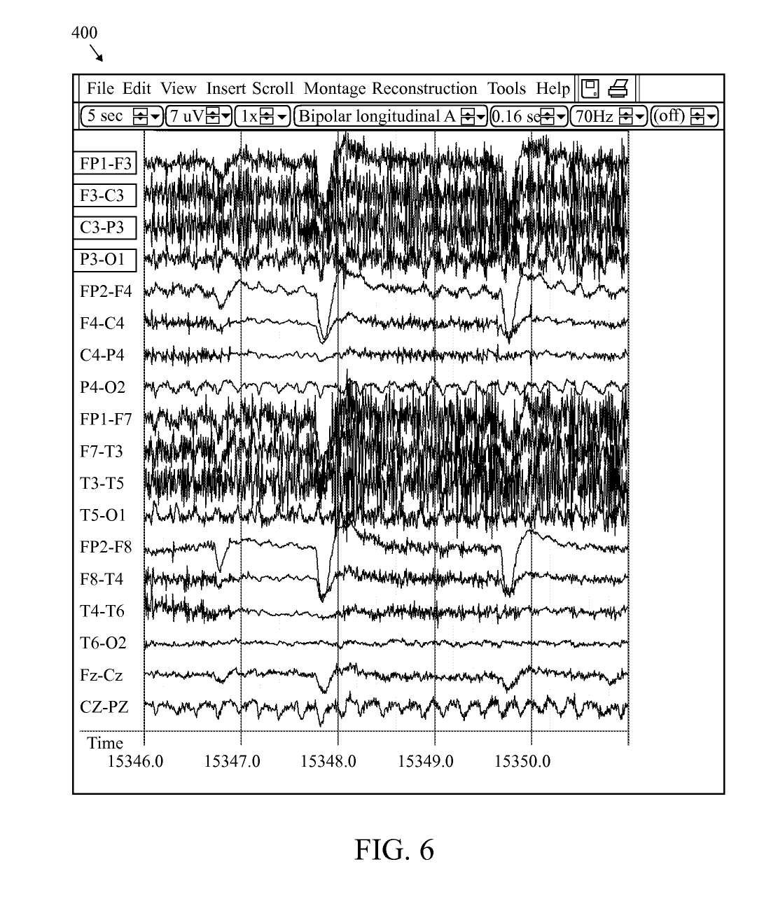

A patient has a plurality of electrodes attached to the patient's head with wires from the electrodes connected to an amplifier for amplifying the signal to a processor, which is used to analyze the signals from the electrodes and create an EEG recording. The brain produces different signals at different points on a patient's head. Multiple electrodes are positioned on a patient's head as shown in FIGS. 3 and 4. The CZ site is in the center. For example, Fp1 on FIG. 4 is represented in channel FP1-F3 on FIG. 6. The number of electrodes determines the number of channels for an EEG. A greater number of channels produce a more detailed representation of a patient's brain activity. Preferably, each amplifier 42 of an EEG machine component 40 corresponds to two electrodes 35 attached to a head of the patient 15. The output from an EEG machine component 40 is the difference in electrical activity detected by the two electrodes. The placement of each electrode is critical for an EEG report since the closer the electrode pairs are to each other, the less difference in the brainwaves that are recorded by the EEG machine component 40. A more thorough description of an electrode utilized with the present invention is detailed in Wilson et al., U.S. Pat. No. 8,112,141 for a Method And Device For Quick Press On EEG Electrode, which is hereby incorporated by reference in its entirety.

The EEG is optimized for automated artifact filtering. The EEG recordings are then processed using neural network algorithms to generate a processed EEG recording, which is analyzed for display. During acquisition of the EEG recording, a processing engine performs continuous analysis of the EEG waveforms and determines the presence of most types of electrode artifact on a channel-by-channel basis. Much like a human reader, the processing engine detects artifacts by analyzing multiple features of the EEG traces. The preferred artifact detection is independent of impedance checking During acquisition the processing monitors the incoming channels looking for electrode artifacts. When artifacts are detected they are automatically removed from the seizure detection process and optionally removed from the trending display. This results in much a much higher level of seizure detection accuracy and easier to read trends than in previous generation products.

Algorithms for removing artifact from EEG typically use Blind Source Separation (BSS) algorithms like CCA (canonical correlation analysis) and ICA (Independent Component Analysis) to transform the signals from a set of channels into a set of component waves or “sources.”

In one example an algorithm called BSS-CCA is used to remove the effects of muscle activity from the EEG. Using the algorithm on the recorded montage will frequently not produce optimal results. In this case it is generally optimal to use a montage where the reference electrode is one of the vertex electrodes such as CZ in the international 10-20 standard. In this algorithm the recorded montage would first be transformed into a CZ reference montage prior to artifact removal. In the event that the signal at CZ indicates that it is not the best choice then the algorithm would go down a list of possible reference electrodes in order to find one that is suitable.

It is possible to perform BSS-CCA directly on the user-selected montage. However this has two issues. First this requires doing an expensive artifact removal process on each montage selected for viewing by the user. Second the artifact removal will vary from one montage to another, and will only be optimal when a user selects a referential montage using the optimal reference. Since a montage that is required for reviewing an EEG is frequently not the same as the one that is optimal for removing artifact this is not a good solution.

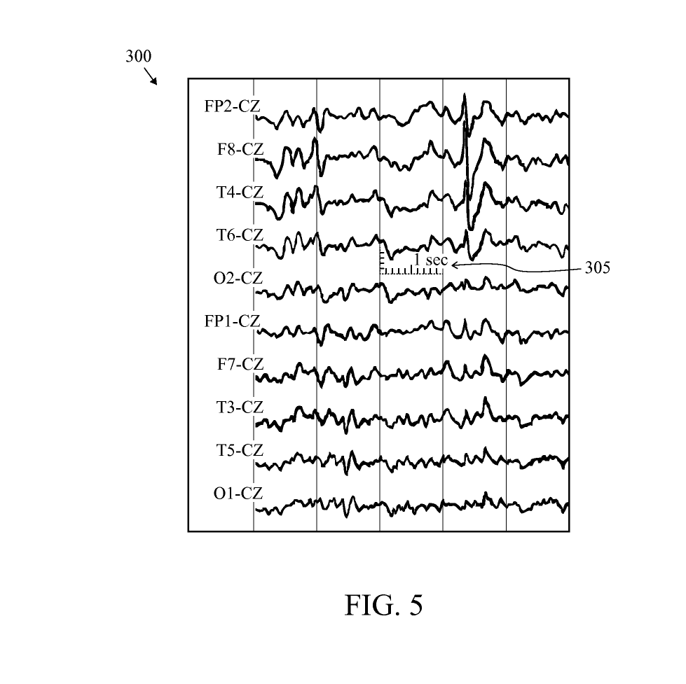

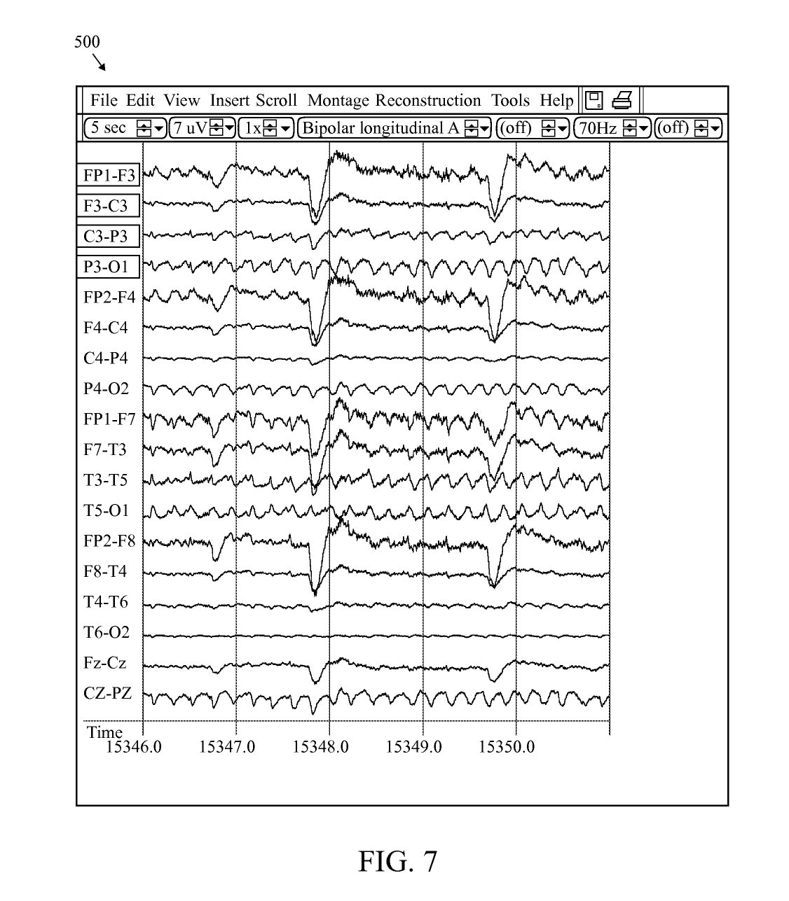

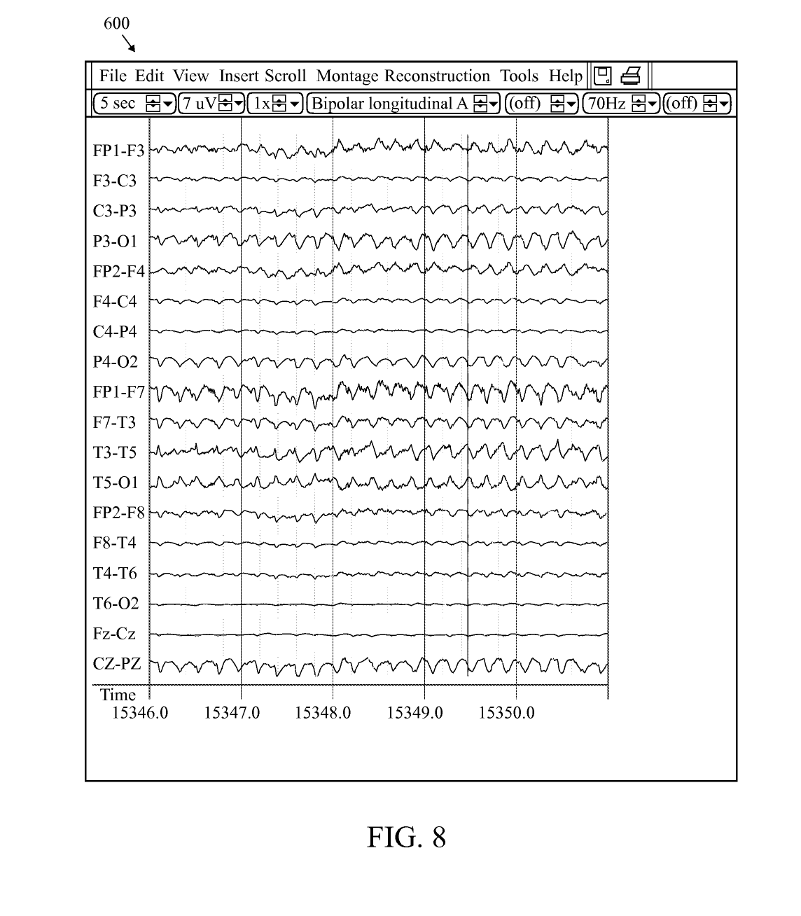

The FIGS. 5-8 illustrate how removing artifacts from the EEG signal allow for a clearer illustration of a brain's true activity for the reader. FIG. 6 is an illustration of an EEG recording 4000 containing a seizure, a muscle artifact and an eye movement artifact. FIG. 7 is an illustration of the EEG recording 5000 of FIG. 6 with the muscle artifact removed. FIG. 8 is an illustration of the EEG recording 6000 of FIG. 7 with the eye movement artifact removed.

Various trends for an EEG recording are generated by a processing engine. A seizure probability trend, a rhythmicity spectrogram, left hemisphere trend, a rhythmicity spectrogram, right hemisphere trend, a FFT spectrogram left hemisphere trend, a FFT spectrogram right hemisphere trend, an asymmetry relative spectrogram trend, an asymmetry absolute index trend, an aEEG trend, and a suppression ration, left hemisphere and right hemisphere trend.

Rhythmicity spectrograms allow one to see the evolution of seizures in a single image. The rhythmicity spectrogram measures the amount of rhythmicity which is present at each frequency in an EEG record.

The seizure probability trend shows a calculated probability of seizure activity over time. The seizure probability trend shows the duration of detected seizures, and also suggests areas of the record that may fall below the seizure detection cutoff, but are still of interest for review. The seizure probability trend when displayed along with other trends, provides a comprehensive view of quantitative changes in an EEG.

An additional description of analyzing EEG recordings is set forth in Wilson et al., U.S. patent application Ser. No. 13/684,469, filed on Nov. 23, 2012, for a User Interface For Artifact Removal In An EEG, which is hereby incorporated by reference in its entirety. An additional description of analyzing EEG recordings is set forth in Wilson et al., U.S. patent application Ser. No. 13/684,556, filed on Nov. 25, 2012, for a Method And System For Detecting And Removing EEG Artifacts, which is hereby incorporated by reference in its entirety.

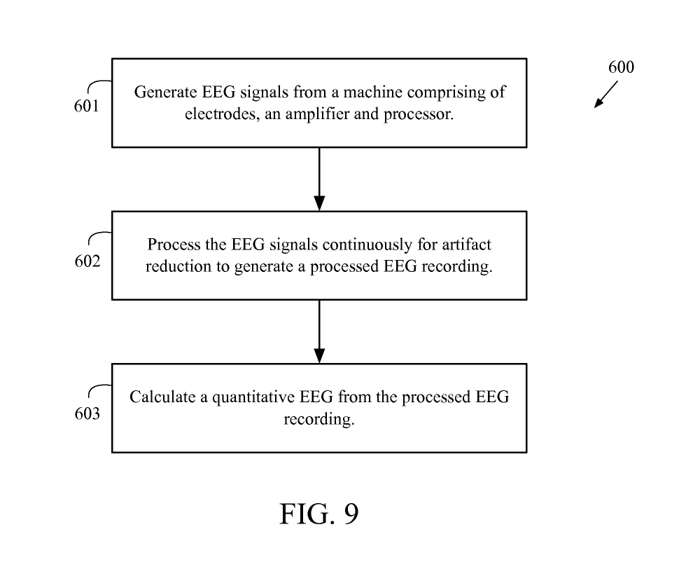

As shown in FIG. 9, a method for calculating a quantitative EEG is generally designated 600. At block 601, EEG signals are generated from an EEG machine comprising a plurality of electrodes, an amplifier and processor. At block 602, the EEG signals are processed continuously for artifact reduction to generate a processed EEG recording. At block 601, a quantitative EEG is calculated from the processed EEG recording. Preferably, Fast Fourier Transform signal processing is used to compute the quantitative EEG. The reduced artifact types are selected from the group comprising an eye blink artifact, a muscle artifact, a tongue movement artifact, a chewing artifact, and a heartbeat artifact.

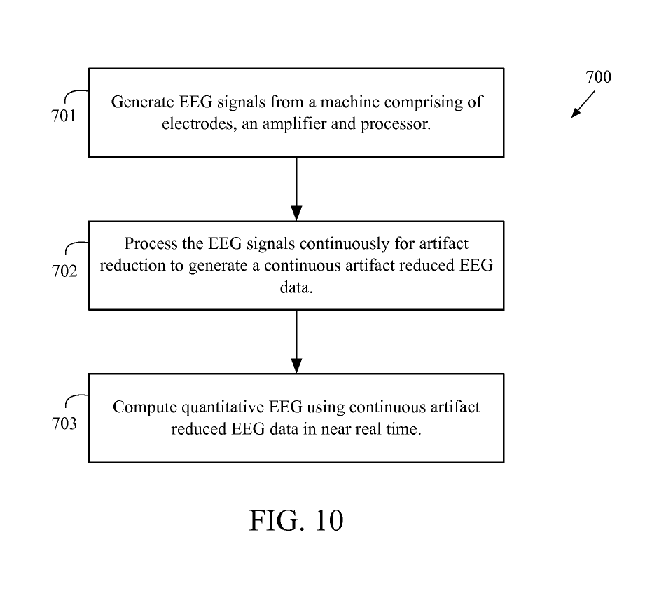

As shown in FIG. 10, method for calculating a quantitative EEG is generally designated 700. At block 701, EEG signals are generated from an EEG machine comprising electrodes, an amplifier and processor. At block 702, the EEG signals are processed continuously for artifact reduction to generate a continuous artifact reduced EEG data. At block 703, a quantitative EEG is computed using continuous artifact reduced EEG data in near real time. The method further includes anticipating a stroke based on the quantitative EEG. The method alternatively includes utilizing the quantitative EEG for seizure detection.

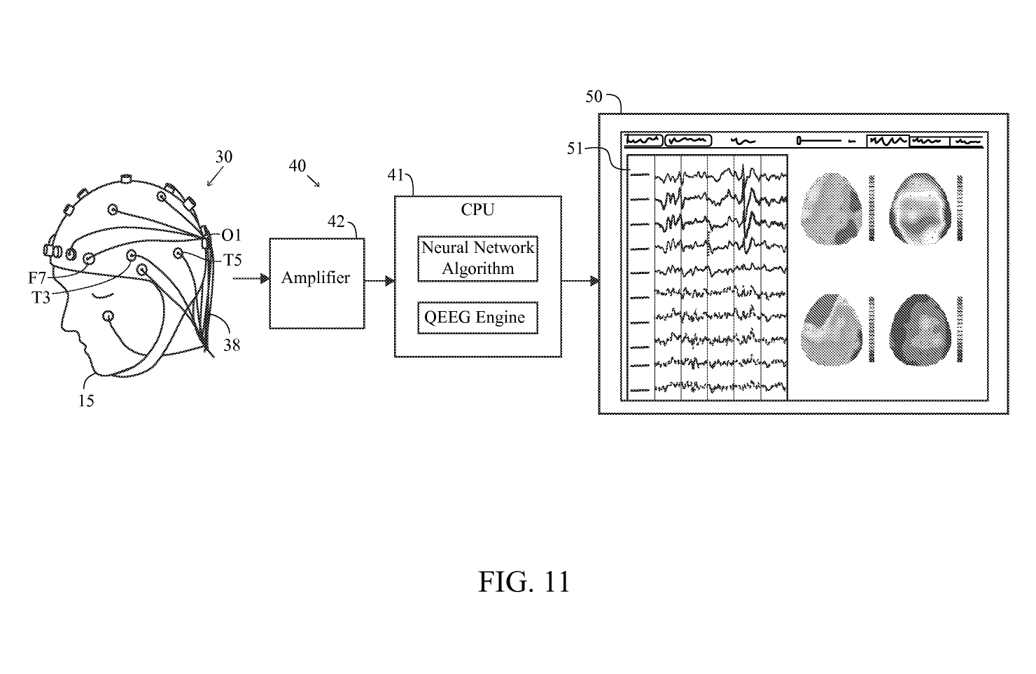

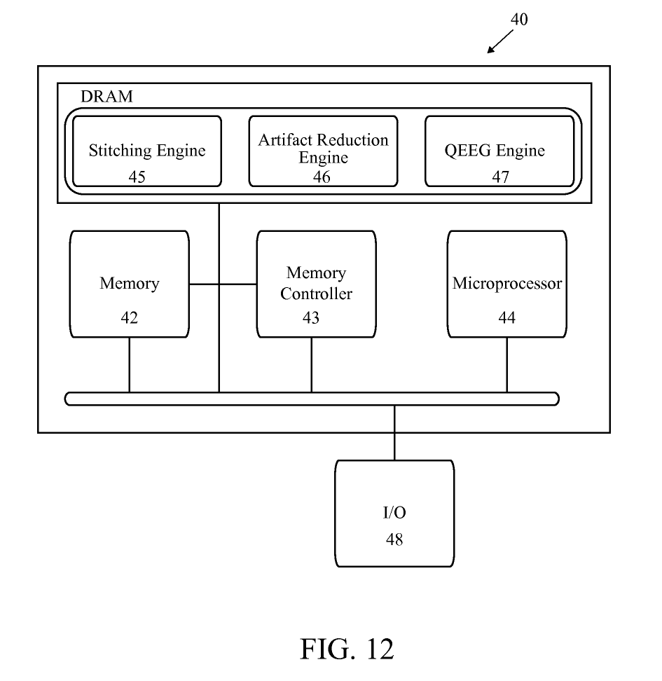

FIGS. 11 and 12 illustrate a system for calculating a quantitative EEG. A patient 15 wears an electrode cap 31, consisting of a plurality of electrodes 35a-35c, attached to the patient's head with wires 38 from the electrodes 35 connected to an EEG machine component 40 which consists of an amplifier 42 for amplifying the signal to a computer 41 with a processor, which is used to analyze the signals from the electrodes 35 and generate an EEG recording and a qEEG 51, which can be viewed on a display 50. The CPU 41 includes a software program for a neural network algorithm and a software program for a qEEG engine. As shown in FIG. 12, an artifact reduction engine, a qEEG engine 47, a microprocessor 44, a memory 42, a memory controller 43 and an I/O 48 ar components of the EEEG machine 40. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is analyzed for display.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention:

1. A method for calculating a quantitative EEG, the method comprising:
generating a plurality of EEG signals from a machine comprising a plurality of electrodes, an amplifier and processor;
processing the plurality of EEG signals continuously for artifact reduction to generate a processed EEG recording; and
calculating a quantitative EEG from the processed EEG recording.

2. The method according to claim 1 wherein Fast Fourier Transform signal processing is used to compute the quantitative EEG.

3. The method according to claim 1 wherein the reduced artifact types are selected from the group comprising an eye blink artifact, a muscle artifact, a tongue movement artifact, a chewing artifact, and a heartbeat artifact.

4. A system for calculating a quantitative EEG, the system comprising:
a plurality of electrodes for generating a plurality of EEG signals;
a processor connected to the plurality of electrodes to generate an EEG recording from the plurality of EEG signals; and
a display connected to the processor for displaying an EEG recording;
wherein the processor is configured calculate a quantitative EEG from the processed EEG recording.

5. The system according to claim 4 wherein the processor is configured to process the EEG signals with a plurality of neural network algorithms to create the processed EEG recording.

6. The system according to claim 5 wherein the reduced artifact types are selected from the group comprising an eye blink artifact, a muscle artifact, a tongue movement artifact, a chewing artifact, and a heartbeat artifact.

7. A method for calculating a quantitative EEG, the method comprising:
generating a plurality of EEG signals from a machine comprising a plurality of electrodes, an amplifier and processor;
processing the plurality of EEG signals continuously for artifact reduction to generate a continuous artifact reduced EEG data; and
computing quantitative EEG using continuous artifact reduced EEG data in near real time.

8. The method according to claim 7 further comprising anticipating a stroke based on the quantitative EEG.

9. The method according to claim 7 wherein Fast Fourier Transform signal processing is used to compute the quantitative EEG.

10. The method according to claim 7 further comprising utilizing the quantitative EEG for seizure detection.
